(19) United States
(12) Patent Application Publication
(10) Pub. No.: US 2021/0403849 A1
(43) Pub. Date: Dec. 30, 2021

(54) CELLULAR OBJECT GROWTH PLATFORM

(71) Applicant: HEWLETT-PACKARD DEVELOPMENT COMPANY, L.P., Spring, TX (US)

(72) Inventors: Viktor Shkolnikov, Palo Alto, CA (US); Daixi Xin, Palo Alto, CA (US); Yang Lei, Palo Alto, CA (US)

(21) Appl. No.: 16/606,742

(22) PCT Filed: Apr. 27, 2018
(86) PCT No.: PCT/US2018/030035

§ 371 Date: Oct. 19, 2019

(51) Int. Cl. C12M 1/00; C12M 1/42; C12M 1/34; C12N 5/00; C12M 3/06

(52) U.S. Cl. CPC C12M 29/00; C12M 35/04; C12M 41/44; C12M 23/22; C12N 2513/00; C12N 5/0062; C12M 35/02; C12M 23/16; C12M 41/34

(57) ABSTRACT

A cellular object imaging system may include a growth platform. The growth platform may include a body having a cellular fluid suspension region, a media supply passage extending within the body to the cellular object fluid suspension region, at least one fluid pump on the body to selectively deliver media to the cellular object fluid suspension region, a waste discharge passage extending within the body from the cellular object fluid suspension region and a cellular object rotator on the body adjacent the cellular object fluid suspension region to rotate a cellular object within the cellular object fluid suspension region. The cellular object fluid suspension region permits optical imaging of the cellular object suspended in a fluid during rotation by the cellular object rotator.

### BACKGROUND

3D cultures are cells grown in droplets or hydrogels that mimic a physiologically relevant environment. Organoids are miniature organs grown in a lab derived from stem cells and clusters of tissue, wherein the specific cells mimic the function of the organ they model. 3-D cultures and organoids may be used to study basic biological processes within specific organs or to understand the effects of particular drugs. 3-D cultures and organoids may provide crucial insight into mechanisms of cells and organs in a more native environment.

### BRIEF DESCRIPTION OF THE DRAWINGS

FIG. 1 is a schematic diagram illustrating portions of an example cellular object growth platform.

FIG. 2 is a flow diagram of an example cellular object growth and imaging method 100.

FIG. 3 is a schematic diagram illustrating portions of an example cellular object imaging system.

FIG. 4 is a top view illustrating portions of an example cellular object imaging system.

FIG. 5 is a sectional view of the example cellular object imaging system of FIG. 4.

FIG. 6 is a schematic diagram illustrating portions of an example cellular object imaging system.

FIG. 7 is a diagram illustrating portions of the example cellular object imaging system of FIG. 6 illustrating the application of a nonrotating nonuniform electric field to rotate an example cellular object.

FIG. 8 a flow diagram of an example three-dimensional volume modeling method.

FIG. 9 is a diagram schematically illustrating the capture of two-dimensional image frames of a rotating object at different angles.

FIG. 10 is a diagram depicting an example image frame including the identification of features of an object at a first angular position.

FIG. 11 is a diagram depicting an example image frame including the identifications of the features of the object at a second different angular position.

FIG. 12 is a diagram illustrating triangulation of the different identified features for the merging and alignment of features from the frames.

FIG. 13 is a diagram illustrating an example three-dimensional volumetric parametric model produced from the example image frames of FIGS. 9 and 10.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed are systems and methods that utilize a cellular object growth platform to facilitate the growth of 3D cell cultures and organoids (collectively referred to as cellular objects). The disclosed systems and methods grow such cellular objects on a growth platform such as a microfluidic chip. The disclosed systems and methods grow such cellular objects on a growth platform that may additionally serve as a platform for analysis of the cellular objects. For example, the growth platform may facilitate rotation of such cellular objects for three-dimensional imaging or model reconstruction. Because the growth platform facilitates both growth and analysis on a single platform, multiple cellular object may be grown at once and multiple different studies may be concurrently performed to reduce cost and increase the amount of useful information that may be derived from such experiments or analysis.

Disclosed herein are example cellular object imaging systems may include a growth platform. The growth platform may include a body having a cellular fluid suspension region, a media supply passage extending within the body to the cellular object fluid suspension region, at least one fluid pump on the body to selectively deliver media to the cellular object fluid suspension region, a waste discharge passage extending within the body from the cellular object fluid suspension region and a cellular object rotator on the body adjacent the cellular object fluid suspension region to rotate a cellular object within the cellular object fluid suspension region. The cellular object fluid suspension region permits optical imaging of the cellular object suspended in a fluid during rotation by the cellular object rotator. In one implementation, the at least one fluid pump may push media within media supply passage to the cellular object fluid suspension region in another implementation, the at least one fluid pump may draw media within media supply passage into the cellular object fluid suspension region.

Disclosed herein is cellular object growth and imaging method that may include providing a cellular object fluid suspension region of a growth platform with a cellular object. A media, the environment and nutrients for the cellular object is controllably pumped to the cellular object fluid suspension region with a first pump on the growth platform. Waste may further be controllably pumped from the cellular object fluid suspension region. The cellular object is rotated within the cellular object fluid suspension region while the cellular object is imaged during its rotation.

Disclosed herein is an example non-transitory computer-readable medium that contains instructions to direct a processing unit to output control signals to a pump on a cellular object growth platform to supply fluid media to a cellular object fluid suspension region of the growth platform and to output control signals to cause waste to be discharged from the cellular object fluid suspension region. The instructions further direct the processing unit to output control signals to electrodes on the growth platform to form an electric field within the cellular object fluid suspension region to rotate the cellular object for imaging.

In some implementations, the growth platform may be formed as a microfluidic chip having microfluidic passages or channels through which media, growth media nutrients, are supplied to at least one growing cellular object and through which waste from the at least one growing cellular object is discharged. The fluid passages, such as microfluidic passages, may facilitate conveyance of different fluids (e.g., liquids having different chemical compounds, different physical properties, different concentrations, etc.) to the microfluidic output channel. In some examples, fluids may have at least one different fluid characteristic, such as vapor pressure, temperature, viscosity, density, contact angle on channel walls, surface tension, and/or heat of vaporization. It will be appreciated that examples disclosed herein may facilitate manipulation of small volumes of liquids.

As will be appreciated, examples provided herein may be formed by performing various microfabrication and/or micromachining processes on a substrate to form and/or connect structures and/or components. Substrates forming the various fluidic components may comprise a silicon based wafer or other such similar materials used for microfabricated devices (e.g., glass, gallium arsenide, plastics, etc.). Examples may comprise microfluidic channels, fluid actuators, and/or volumetric chambers. Microfluidic channels and/or chambers may be formed by performing etching, microfabrication processes (e.g., photolithography), or micromachining processes in a substrate. Accordingly, microfluidic channels and/or chambers may be defined by surfaces fabricated in the substrate of a microfluidic device. In some implementations, microfluidic channels and/or chambers may be formed by an overall package, wherein multiple connected package components combine to form or define the microfluidic channel and/or chamber.

In some examples described herein, at least one dimension of a microfluidic channel and/or capillary chamber may be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.). For example, some microfluidic channels may facilitate capillary pumping due to capillary force. In addition, examples may couple at least two microfluidic channels to a microfluidic output channel via a fluid junction.

As used herein, an inertial pump corresponds to a fluid actuator and related components disposed in an asymmetric position in a fluid channel, where an asymmetric position of the fluid actuator corresponds to the fluid actuator being positioned less distance from a first end of the fluid channel as compared to a distance to a second end of the fluid channel. Accordingly, in some examples, a fluid actuator of an inertial pump is not positioned at a mid-point of a fluid channel. The asymmetric positioning of the fluid actuator in the fluid channel facilitates an asymmetric response in fluid proximate the fluid actuator that results in fluid displacement when the fluid actuator is actuated. Repeated actuation of the fluid actuator causes a pulse-like flow of fluid through the fluid channel.

In some examples, an inertial pump includes a thermal actuator having a heating element (e.g., a thermal resistor) that may be heated to cause a bubble to form in a fluid proximate the heating element. In such examples, a surface of a heating element (having a surface area) may be proximate to a surface of a fluid channel in which the heating element is disposed such that fluid in the fluid channel may thermally interact with the heating element. In some examples, the heating element may comprise a thermal resistor with at least one passivation layer disposed on a heating surface such that fluid to be heated may contact a topmost surface of the at least one passivation layer. Formation and subsequent collapse of such bubble may generate flow of the fluid. As will be appreciated, asymmetries of the expansion-collapse cycle for a bubble may generate such flow for fluid pumping, where such pumping may be referred to as “inertial pumping.”

In other examples, the fluid actuator(s) forming an inertial pump or used to eject fluid through an ejection orifices or nozzle may comprise piezo-membrane based actuators, electrostatic membrane actuators, mechanical/impact driven membrane actuators, magnetostrictive drive actuators, electrochemical actuators, external laser actuators (that form a bubble through boiling with a laser beam), other such microdevices, or any combination thereof. In some implementations, the fluid actuators may displace fluid through movement of a membrane (such as a piezo-electric membrane) that generates compressive and tensile fluid displacements to thereby cause inertial fluid flow.

As will be appreciated, the fluid actuator forming the inertial pump may be connected to a controller, and electrical actuation of the fluid actuator by the controller may thereby control pumping of fluid. Actuation of the fluid actuator may be of relatively short duration. In some examples, the fluid actuator may be pulsed at a particular frequency for a particular duration. In some examples, actuation of the fluid actuator may be 1 microsecond (μs) or less. In some examples, actuation of the fluid actuator may be within a range of approximately 0.1 microsecond (μs) to approximately 10 milliseconds (ms). In some examples described herein, actuation of the fluid actuator includes electrical actuation. In such examples, a controller may be electrically connected to a fluid actuator such that an electrical signal may be transmitted by the controller to the fluid actuator to thereby actuate the fluid actuator. Each fluid actuator of an example microfluidic device may be actuated according to actuation characteristics. Examples of actuation characteristics include, for example, frequency of actuation, duration of actuation, number of pulses per actuation, intensity or amplitude of actuation, phase offset of actuation.

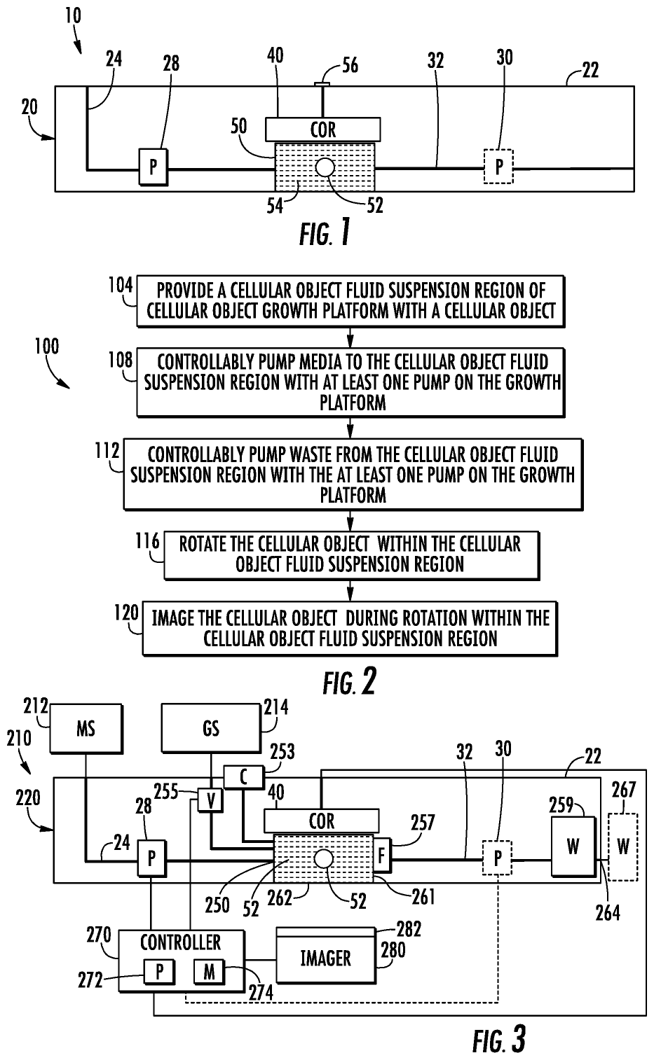

FIG. 1 schematically illustrates portions of an example cellular object imaging system 10. Cellular object imaging system 10 facilitates imaging of cellular objects (3D cell cultures and/or cells). Cellular object imaging system utilizes a cellular object growth platform 20 to both grow a cellular object and stage the cellular object for imaging. In the example illustrated, the cellular object growth platform 20 facilitates rotation of the cellular object for three-dimensional image reconstruction or modeling of the cellular object while the cellular object is still contained or supported by platform 20. Because the cellular object is rotated image while still supported by the growth platform 20, handling steps, cost and process complexity are reduced. Cellular object growth platform 20 comprises body 22, media supply passage 24, at least one fluid pump 28, waste discharge passage 32 and cellular object rotator (COR) 40.

Body 22 comprises a structure supporting or containing the remaining components of platform 20. In the example illustrated, body 22 comprises a cellular object fluid suspension region 50. Cellular object fluid suspension region 50 contains at least one cellular object during its rotation by cellular object rotator 40. Cellular object fluid suspension region 50 comprise at least one transparent or translucent portion through which images of the rotating cellular object may be captured by an imaging system.

In one implementation, cellular object fluid suspension region 50 comprises an opening in the form of an imaging window having a transparent or translucent pane across the opening and through which imaging of the rotating cellular object occurs. In one implementation, at least the translucent or transparent pane maybe a gas permeable, facilitating exchange as air or other non-air gas through the pain with the fluid and cellular object contained in chamber 50. In another implementation, cellular object fluid suspension region 50 comprises a fluid chamber having an opening through which imaging of the rotating cellular object occurs. In such an implementation, surface tension of the fluid across the opening retains the fluid and the suspended cellular object within the chamber. In such an implementation, gas may be exchanged with respect to the fluid or the cellular object within chamber 50 through the opening.

In still another implementation, cellular object fluid suspension region 50 may comprise an open space adjacent an opening or multiple openings which are sized and located such that fluid may pass through such openings and be suspended from such openings as a pendant drop of fluid which projects from or is suspended from body 22. In such an implementation, the drop of fluid forms or defines the cellular object fluid dispensing region 50 and contains at least one cellular object as the at least one cellular object is rotated. In such an implementation, the cellular object is contained by the walls of the fluid drop. In some implementations, the cellular object may be injected into the pendant drop.

Media supply passage 24 supplies nutrients or other growing medium for a cellular object (referred to as “media”) to cellular object fluid suspension region. Media supply passage 24 may comprise a microfluidic channel or passage formed and extending within body 22 and connected to cellular object fluid suspension region 50. In one implementation, media supply passage 24 may be connected to a reservoir of media on body 22. In one implementation, media supply passage 24 maybe connected to a port or other interface for connection to an external supply of media.

Fluid pump 28 is situated along media supply passage 24 on body 22. Fluid pump 28 controllably supplies and drives (pushes) media to cellular object fluid suspension region 50. In one implementation, fluid pump 28 comprises an inertial pump. In one implementation, fluid pump 28 comprises an inertial pump having a fluid actuator in the form of a thermoresistor. In other implementations, inertial pump may utilize other fluid actuators. In other implementations, the fluid pump 28 may comprise other types of fluid displacement devices or pumps. In some implementation, fluid pump 28 may additionally comprise valves to control the flow of media to cellular object fluid suspension region 50. For example, in one implementation, one-way valves may be employed to control the flow of media to region 50.

Waste discharge passage 32 extends from region 50. Waste discharge passage 32 facilitates the discharge of waste and other contaminants/fluids from region 50. In one implementation, waste discharge passage 32 is sized so as to inhibit the cellular object or those cellular objects of interest being grown, rotated and analyzed in region 50 from entering fluid discharge region 32 and being unintentionally discharged along with waste. In other implementations, the filtering mechanism may be employed between region 50 and fluid discharge passage 32 inhibit the accidental entry of the cellular object into waste discharge passage 32. In one implementation, waste discharge passage 32 is connected to a waste reservoir provided on or in body 22. In another implementation, waste discharge passage 32 is connected to a port or other fluid coupling interface to facilitate connection to an external waste reservoir or receiver.

In one implementation, waste within region 50 is moved into waste discharge passage 32 through pressure differentials created by fluid pump 28. As shown by broken lines, in some implementations, cellular object growth platform may additionally comprise pump 30. Pump 30 is situated along waste discharge passage 32. Pump 30 controllably pumps and drives waste along waste discharge patch and 32 to draw waste out of region 50. In one implementation, pump 30 comprises an inertial pump. In one implementation, fluid pump 28 comprises an inertial pump having a fluid actuator in the form of a thermoresistor. In other implementations, inertial pump may utilize other fluid actuators. In other implementations, the fluid pump 28 may comprise other types of fluid displacement devices or pumps. In some implementations, fluid pump 30 may be provided while fluid pump 28 is omitted. In such an implementation, in addition to ejecting waste from region 50, fluid pump 30 also draws media along media supply passage 24 into region 50.

Cellular object rotator 40 comprises a device to controllably rotate a cellular object, such as cellular object 52 (schematically shown) while the cellular object 52 is suspended in a fluid 54. In one implementation, cellular object rotator 40 provides electro-kinetic rotation. In one implementation, cellular object rotator 40 utilizes electrodes which form an electric field through and across region 50, wherein the electric field causes rotation of cellular object 52. In one implementation, cellular object rotator 40 comprises a pair of electrodes that apply a nonrotating nonuniform electric field so as to apply a dielectrophoretic torque to cellular object 52 is to rotate cellular object 52 while cellular object 52 is suspended in fluid 54. In one implementation, body 22 of platform 20 is generally planar, extending in a flat plane, wherein cellular object rotator 40 rotate the cellular object 52 about an axis parallel to the plane. Such rotation facilitates the capturing of images of the cellular object 52 at different angles to facilitate three-dimensional reconstruction or modeling of cellular object 52 for analysis. In the example illustrated, cellular object rotator 40 may have electrical contact pads 56 four electrical connection to a remote controller. In other implementations, such communication with a remote controller may be wireless. In yet other implementations, the controller may be local, supported by body 22.

FIG. 2 is a flow diagram of an example cellular object growth and imaging method 100. Method 100 facilitates the growth and imaging of a cellular object on a single platform. Although method 100 is described in the context of being carried out using growth platform 20, it should be appreciated that method 100 may likewise be carried out using any of the growth platforms and imaging systems described hereafter or using similar growth platforms or imaging systems.

As indicated by block 104, the cellular object fluid suspension region 50 of growth platform, such as platform 20, is provided with a cellular object, such as cellular object 52. In one implementation, the cellular object may be injected, along with surrounding fluid, with a syringe into the cellular object fluid suspension region. In another implementation, cellular object may be directed into region 50 through one or more passages provided on the growth platform. In some implementations, multiple cellular objects may be provided in region 50.

As indicated by block 108, media is controllably pumped or supplied to the cellular object fluid suspension region with at least one pump on a cellular object growth platform. The media comprises the culture of fluid environment for the cellular object and may include nutrients for growth of the cellular object. In one implementation, the at least one pump may comprise a fluid pump located along a media supply passage, such as pump 28 located along media supply passage, wherein the pump pumps or pushes fluid into the cellular object fluid suspension region. In another implementation, the at least one pump may comprise a fluid pump located along a fluid discharge passage, wherein the pump draws the media within a media supply passage into the cellular object fluid suspension region. In yet another implementation, a pump located along a fluid supply passage and a pump located along a fluid discharge passage may be used to concurrently push media into the region as well as draw media into the region.

As indicated by block 112, waste is controllably pumped from the cellular object fluid suspension region with the at least one pump on the growth platform. In one implementation, the waste is controllably pumped using a pump, such pump 30, located along the waste discharge passage. In another implementation, the waste is controllably pumped using a pump, such as pump 28 located along media supply passage 24, wherein the pump drives media into the region and the existing fluid within the region pushes or dispels waste into and along the fluid discharge passage. In yet another implementation, pumps located along both the media supply passage and the waste discharge passage and a work in combination to pump waste from the cellular object fluid suspension region.

As indicated by block 116, cellular object rotator 40 is actuated so as to rotate the cellular object within the cellular object fluid suspension region. In one implementation, such rotation is in a controlled fashion at a controlled revolution speed to facilitate the capture of images of the cellular object at predetermined angular positions. In one implementation, electro-kinetic rotation is used to rotate the cellular object.

In one implementation, the cellular object 52 is rotated with electrodes that form an electric field through and across the cellular object fluid suspension region, wherein the electric field causes rotation of cellular object. In one implementation, a pair of electrodes apply a nonrotating nonuniform electric field so as to apply a dielectrophoretic torque to cellular object to the rotate the cellular object while cellular object is suspended in fluid. In one implementation, body of the growth platform is generally planar, extending in a flat plane, wherein the cellular object 52 is rotated about an axis parallel to the plane. Such rotation facilitates the capturing of images of the cellular object at different angles to facilitate three-dimensional reconstruction or modeling of cellular object for analysis.

As indicated by block 120, the cellular object is imaged during rotation of the cellular object while the cellular object suspended in fluid within the cellular object fluid suspension region. In one implementation, multiple images of the cellular object are captured at different angular positions of the cellular object. As will be described hereafter, triangulation may be utilized on distinct points in the images and a three-dimensional reconstruction or three-dimensional model of the cellular object may be formed and stored for analysis.

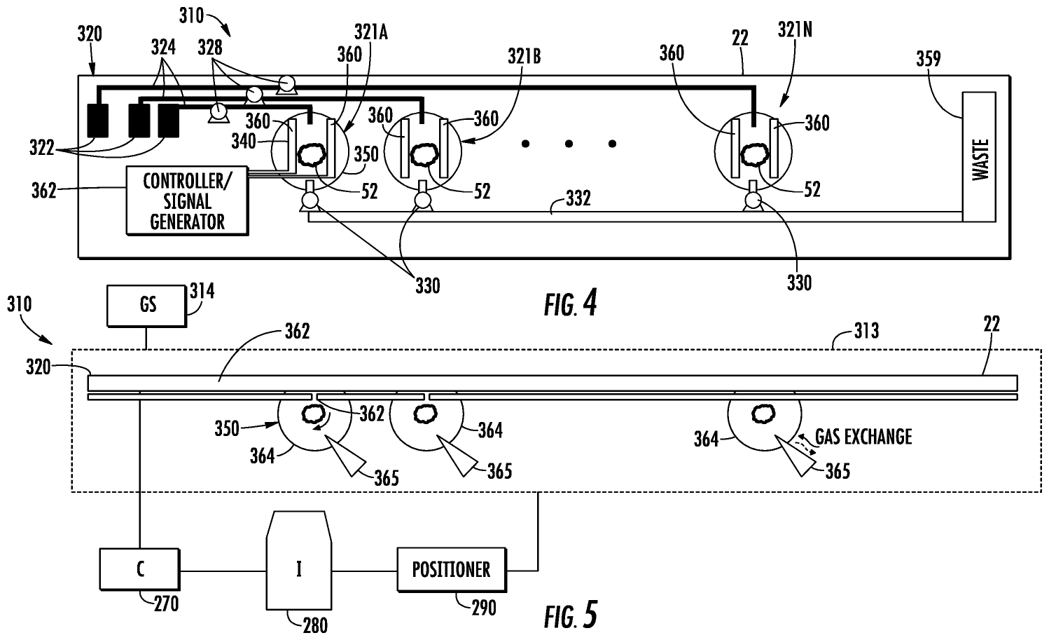

FIG. 3 is a schematic diagram illustrating portions of an example cellular object imaging system 210. Cellular object imaging system 210 comprises media supply 212, gas supply 214, growth platform 220, controller 270 and imager 280. Media supply 212 comprises a supply of media for the cellular object 52 being grown or maintained for analysis. The media supplied by media supply 212 may comprise chemical compositions or other materials providing the environment for the growth or maintenance of the cellular object 52. Examples of media that may be supplied include, but are not limited to, standard tissue culture media like DMEM and RPMI consisting of crucial nutrients such as glucose and serum. In the example illustrated, media supply 212 comprises a remote supply releasably connected to growth platform 220 by a plug, port, connector or other interface. In other implementations, display 212 may be provided on growth platform 220.

Gas supply 214 comprises a supply of gas for the cellular object 52 being grown or maintained for analysis. The gas supplied by gas supply 214 may comprise air or may comprise non-air gaseous compositions providing the environment for the growth or maintenance of the cellular object 52. Examples of gases that may be supplied include, but are not limited to, 5% carbon dioxide. In the example illustrated, media supply 212 comprises a remote supply releasably connected to growth platform 220 by a plug, port, connector or other interface. In other implementations, display 212 may be provided on growth platform 220.

Growth platform 220 provides a single structure or body facilitating both the growth and maintenance of cellular objects as well as their rotation for imaging and analysis. In one implementation, growth platform 220 may comprise a microfluidic chip having microfluidic fluid passages. Growth platform 220 is similar to growth platform 20 described above except that growth platform 220 is additionally illustrated as specifically comprising cellular object fluid suspension region 250, cellular object injection port 253, gas supply valve 255, filter 257 and waste reservoir 259. Those remaining components or elements of growth platform 220 which correspond to components or elements of growth platform 20 are numbered similarly.

Cellular object fluid suspension region 250 is similar to region 50 except that region 250 is specifically illustrated as comprising a fluid chamber having an opening or window 261 covered by a translucent or transparent windowpane 262. Windowpane 262 facilitates imaging of cellular object 52 while cellular object 52 is being rotated within region 250 during his rotation by cellular object rotator 40. In one implementation, the chamber forming region 250 is enlarged relative to one or both of media supply passage 24 and media discharge passage 32. In another implementation, the chamber forming region 250 comprises an intermediate fluid passage of generally the same size extending between filter 257 and media supply passage 24.

Cellular object injection port 253 comprises a port or structure through which cellular object 252 may be injected or placed within region 250. In one implementation, cellular object four comprises a port connected to a fluid passage extending to region 250. In yet another implementation, cellular object injection port 253 may comprise a membrane through which a needle of the syringe may be inserted to inject a cellular object into region 250.

Gas supply valve 255 comprise a valve mechanism formed in body 22 so as to control the supply of gas from the gas supply 214 to region 250. In some implementations, gas supply valve 255 may be omitted.

Filter 257 comprises a structure that retains so object 52 within the interior of region 250. In one implementation, filter 252 may comprise at least one pillar formed in body 22, wherein the at least one pillar facilitates the passage of waste into media discharge passage 32 of blocking passage of cellular object 52. In another implementation, filter 252 comprises a mesh-like material or other filtering mechanism. In some implementations, filter 257 may be omitted, wherein the reduced size or dimensioning of waste discharge passage 32 retains cellular object 52 within region 250.

Waste reservoir 259 comprise the chamber formed within body 22 that contains and at least temporarily stores waste received through waste discharge passage 32. In one implementation, with River 259 may be connected to an external port 2642 facility the discharge waste from reservoir 259. As indicated by broken lines, in some implementations, waste reservoir 259 may be omitted, such as where an external waste reservoir 267 is connectable to port 264 to receive waste directly from waste discharge passage 32.

Controller 270 comprises a processing unit 272 in a non-transitory computer-readable medium in the form of memory 274. Processing unit 272 follows instructions contained in memory 274. Memory 274 contains instructions that direct processing unit 272 to control the operation of pumps 28 and 30, gas supply valve 255 (when provided), core object rotator 40 and imager 280. For example, controller 270 outputs control signals controlling pump 28 and pump 30 to control the supply of media to region 250 and the discharge of waste from region 250. Controller 270 may additionally control valve 255 to control the supply of gas from gas supply 214. Controller 270 may further control cellular object rotator 40. Controller 270 may control the rate at which so the object 52 is rotated during imaging.

Imager 280 captures images of the rotating cellular object 52 at different angular positions during his rotation to facilitate subsequent three-dimensional image reconstruction of the cellular object 52 as will be described hereafter. In one implementation come imager 280 may comprise a camera having an optical lens 282 facility microscopic viewing and imaging of cellular object 52. In some implementations, system 210 may comprise multiple imagers 280, multiple sets of cameras, positioned so as to concurrently capture images of cellular object 52 as it is being rotated.

FIGS. 4 and 5 schematically illustrate an example cellular object imaging system 310. As shown by FIG. 5, cellular object imaging system 310 comprises gas exchange chamber 313, gas supply 314, growth platform 320, controller 270, imager 280 and positioner 290. Gas exchange chamber 313 comprise a chamber by which gas is contained and supplied to facilitate the growth or maintenance of cellular objects being analyzed. In the example illustrated, gas exchange chamber 313 removably receives growth platform 320. In one implementation, gas exchange chamber 310 comprises at least portions that are translucent or transparent to facilitate the imaging of cellular objects as such objects are being rotated. In other implementations, growth platform 320 withdrawn from chamber 313 when such imaging is performed.

Gas supply 314 is similar to gas supply 214 described above. Gas supply 314 comprises a supply of a gas, such as air or a non-gas to the interior of chamber 313, wherein the supply gas is exchanged with respect to the region containing the cellular object being grown or maintained. Examples of gases that may be supplied include, but are not limited to, 5% carbon dioxide.

Cellular object growth platform 320 provides a single structure or body facilitating both the growth and maintenance of cellular objects as well as their rotation for imaging and analysis. In one implementation, growth platform 320 may comprise a microfluidic chip having microfluidic fluid passages. In the example illustrated, cellular object growth platform 320 provides multiple cellular object growth and imaging units 321A, 321B, . . . 321N (collectively referred to as units 321) facilitating the concurrent growth and imaging of multiple different cellular objects and/or facilitating the concurrent growth of multiple different (or similar) cellular object in different growth or maintenance environments using different media or other different environmental conditions. Cellular platform 320 may facilitate a larger number of tests and data acquisition in a shorter amount of time.

As shown by FIG. 4, a top view of growth platform 320, each of cellular object growth and imaging units 321 comprises an assigned media input 322, an assigned media supply passage 324, an assigned media supply fluid pump 328, an assigned cellular object fluid suspension region 350, a shared waste discharge passage 332, an assigned media discharge pump 330, a shared waste reservoir 359, and an assigned a cellular object rotator 340. Each of such components is formed on body 22, which in some implementations may form a microfluidic chip.

Each media input 322 supplies a media for the cellular object 52 being grown or maintained and imaged. In one implementation, each media input 322 comprises separate port by which media may be supplied to growth platform 320. In some implementations, each media input 322 comprises a reservoir to store and contain such media. In one implementation, each media input 322 stores or receives different media having different properties to facilitate different environments for the growth and maintenance of cellular objects 52.

Media supply passages 324 and pumps 328 are similar to media supply passage 24 and pump 28 described above. In one implementation come media supply passages 324 may comprise microfluidic channels or passages. In one implementation, pump 328 may comprise inertial pumps, such as inertial pumps provided by fluid actuators in the form of thermal resistors. In yet other implementations, pumps 328 may utilize other types of fluid actuators or other types of pumps which are formed in or on body 22 as part of platform 320.

Cellular object fluid suspension regions 350 comprise an open space adjacent to an opening or adjacent to multiple openings 362 (shown in FIG. 5) which are sized and located such that fluid may pass through such openings and be suspended from such openings as a pendant drop 364 of fluid which projects from or is suspended from body 22. In such an implementation, the drop of fluid forms or defines the cellular object fluid dispensing region 350 and contains at least one cellular object as the at least one cellular object is rotated. In such an implementation, the cellular object is contained by the walls of the fluid drop. In some implementations, the cellular object may be injected into the pendant drop with a surrender other insertion device 365 (schematically shown). Such injection may occur prior to insertion of growth platform 320 into chamber 313. Following imaging of the cellular object, or when the analysis of cellular object 52 on growth platform 320 is been completed, the cellular object 52 may be withdrawn using a syringe other withdrawal device in a similar manner. For example, the cellular object 52 may be withdrawn from the pendant drop 364 for further analysis and testing off of growth platform 320.

Waste discharge passage 332 is shared amongst the various units 321. Waste discharge passage 332 directs a flow of waste from each of the unit 3212 a raced reservoir 359. Waste reservoir 359 contains waste on growth platform 320.

In the example illustrated, pumps 330 move waste from their respective regions 350 of units 3212 waste reservoir 359. Each of pumps 330 may be similar to pump 30 described above. In one implementation, each of pumps 330 comprises an inertial pump. In one implementation, the inertial pump utilizes a thermal resistive fluid actuator. In other implementations, each of pump 330 may utilize other fluid actuators as part of an inertial pump or may use other fluid pumps. In some implementations, pump 330 may be omitted.

Cellular object rotators 340 rotate individual cellular objects 52 within their respective regions 350 of unit 321. a device to controllably rotate a cellular object, such as cellular object 52 while the cellular object 52 is suspended in the fluid droplet 364. In one implementation, each cellular object rotator 340 provides electro-kinetic rotation. In one implementation, each cellular object rotator 340 comprises electrodes 360 extending within or adjacent to the respective regions 350. At least one of the electrodes of each of units 321 is electrically charged so as to produce an electric field through and across its respective region, wherein the electric field is manipulator controlled so as to rotate the cellular object 52 suspended in the fluid droplet 364. In one implementation, at least one of the electrodes 360 of each of the units 321 is unlikely charged under the control of a controller/signal generator 362. In one implementation, each unit 321 has a dedicated assigned controller/signal generator. In other implementations, a single controller for a signal generator 362 controls the charging of the electrodes 360 and the rotation of the cellular object 352.

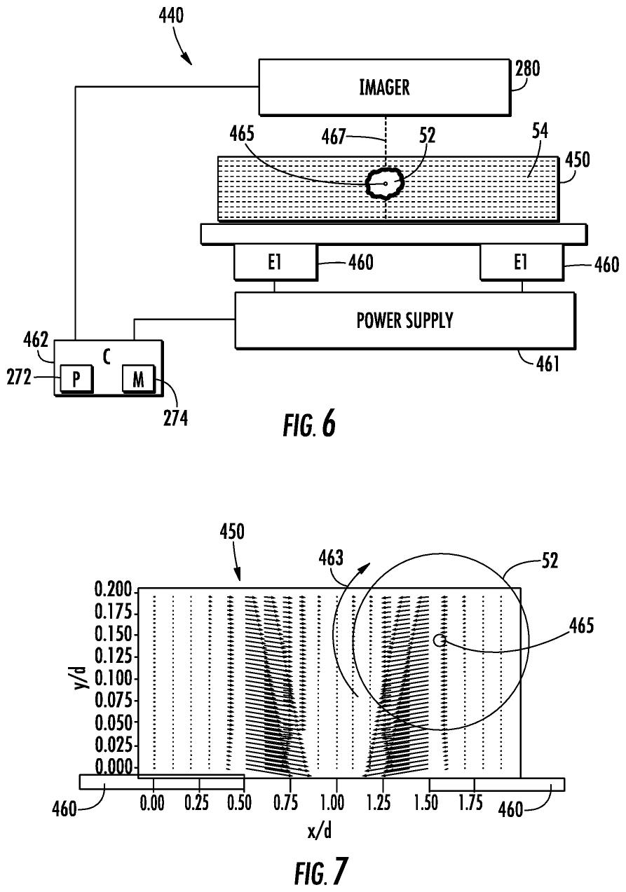

FIGS. 6 and 7 illustrate portions of an example individual cellular object rotator 440 that may be utilized for each of the cellular object rotators 340 in growth platform 320 or for the cellular object rotators 40 in growth platforms 20 and 220 described above. As schematically shown by FIG. 6 cellular object rotator 440 comprises electrodes 460, power supply 461 and controller 462. Electrodes 460 are situated along cellular object fluid suspension region 450 so as to produce an electric field within region 450. In the example growth platform 320, such electrodes 460 may be located within body 22 above or otherwise adjacent the formed fluid droplet 364, wherein electrodes 460 are located in a single plane that is parallel to the plane containing platform 320. In other implementations, electrodes 460 may extend beneath or alongside region 450.

Power supply 461 supply the charged each of electrodes 460 and is under the control of controller 462. Controller 462 is similar to controller 270 described above. Controller 462 comprises a processing unit 272 that follows instructions contained in a non-transitory computer-readable medium in the form of memory 274. Such instructions cause processing unit 272 to control power supply 461 to supply power to electrodes 460 such that electrodes 460 apply a nonrotating nonuniform electric field to cellular object 52 suspended within the fluid 54, wherein nonrotating non-electric field applied to the dielectrophoretic torque to the cellular object 52 so as to rotate the cellular object 52.

In one implementation, the nonrotating nonuniform electric field is an alternating current electric field having a frequency of at least 30 kHz and no greater than 500 kHz. In one implementation, the nonrotating nonuniform electric field has a voltage of at least 0.1 V rms and no greater than 100 V rms. Between taking consecutive images, the cellular object must have rotated a distance that at least equals to the diffraction limit dₗᵢₘ of the imaging optics. The relationship between minimum rotating angle Amin, radius r and diffraction limit distance dₗᵢₘ is θₘᵢₙ=dₗᵢₘ/r. For example, for imaging with light of λ=500 nm and a lens of 0.5 NA, the diffraction limit dₗᵢₘ=λ/(2NA)=500 nm. In the meanwhile, the cellular object cannot rotate too much that there is no overlap between consecutive image frames. So the maximum rotating angle between consecutive images θₘₐₓ=180−θₘᵢₙ. In one implementation, the nonuniform nonrotating electric field produces a dielectrophoretic torque on the cellular object so as to rotate the cellular object at a speed such that the imager 280 may capture images every 2.4 degrees while producing output in a reasonably timely manner. In one implementation where the capture speed of the imager is 30 frames per second, the produced dielectrophoretic torque rotates the cellular object at a rotational speed of at least 12 rpm and no greater than 180 rpm. In one implementation, the produced dielectrophoretic torque rotates the cellular object at least one pixel shift between adjacent frames, but where the picture shift is not so great so as to not be captured by the imager 280. In other implementations, cellular object 52 may be rotated at other rotational speeds.

In one implementation, the rotational axis 465 about which cellular object 52 is rotated extends in the plane of the growth platform (stage) that comprises the cellular object rotator 440. The axis 465 extends perpendicular to the optical axis 467 of imager 280. Because rotation of the cellular object 52 is rotated about axis 465 that is perpendicular to the optical axis 467, imaging of all sides of cellular object 52 at different angular positions is facilitated.

As shown by FIG. 5, controller 270 controls the various operation of growth platform 320 as well as imager 280 and positioner 290. Controller 270 described above with respect to imaging system 210. Controller 270 communicate with the at least one controller/signal generator 362 on growth platform 320 to control the rotation of the cellular object 52. In one implementation, controller 270 may be remote from growth platform 320, communicating with components of growth platform 320 in a wired or wireless fashion. In another implementation, controller 270 may be embodied in or as part of growth platform 320.

Imager 280 is described above with respect to imaging system 210. Imager 280 captures images of the rotating cellular object 52 at different angular positions during his rotation to facilitate subsequent three-dimensional image reconstruction of the cellular object 52 as will be described hereafter. In one implementation come imager 280 may comprise a camera having an optical lens 282 facility microscopic viewing and imaging of cellular object 52. In some implementations, system 210 may comprise multiple imagers 280, multiple sets of cameras, positioned so as to concurrently capture images of cellular object 52 as it is being rotated.

Positioner 290 comprises a device that selectively repositions at least one of growth platform 320 and imager 280 relative to the other. In one implementation, positioner 290 comprises a linear actuator operably coupled to imager 280 to reposition imager 280 opposite to each of the different units 321 for imaging the rotating cellular object 52. In another implementation, positioner 290 comprise a linear actuator operably coupled to growth platform 3202 translate growth platform 320 so as to position each of the fluid droplets 364 containing a cellular object 52 opposite to imager 280. Controller 270 may output control signals to positioner 290 controlling the relative positions of growth platform 320 and imager 280 such that three-dimensional images may be produced for each of the cellular objects 52 in each of the units 321. In other implementations, positioner 290 may be omitted.

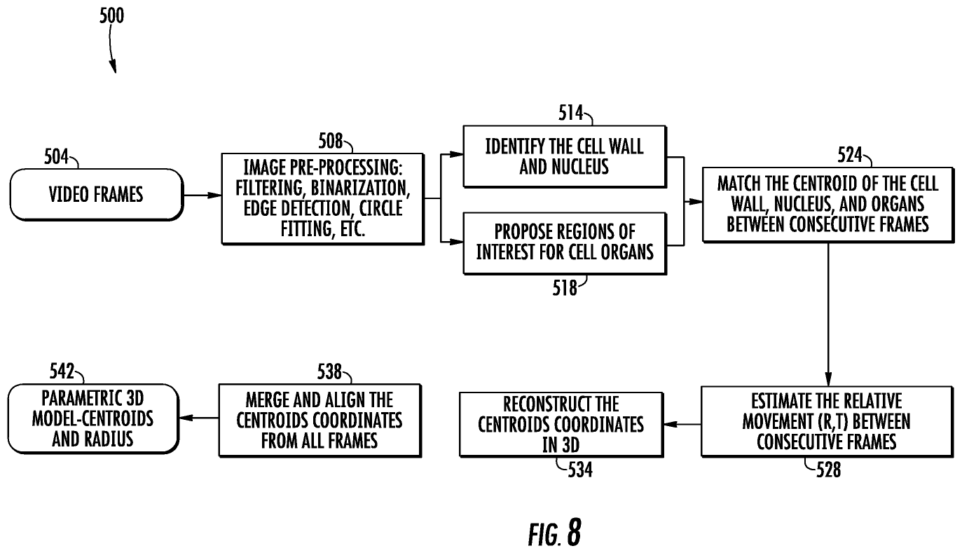

FIG. 8 is a flow diagram of an example three-dimensional volumetric modeling method 500 that may be carried out by controller 470 using captured two-dimensional images of the rotating object 52. As indicated by block 504, a controller, such as controller 470, receives video frames or two-dimensional images captured by the imager/camera 60 during rotation of object 52. As indicated by block 508, various preprocessing actions are taken with respect to each of the received two-dimensional image video frames. Such preprocessing may include filtering, binarization, edge detection, circle fitting and the like.

As indicated by block 514, utilizing such edge detection, circle fitting and the like, controller 470 retrieves and consults a predefined three-dimensional volumetric template of the object 52, to identify various internal structures of the object or various internal points in the object. The three-dimensional volumetric template may identify the shape, size and general expected position of internal structures which may then be matched to those of the two-dimensional images taken at the different angles. For example, a single cell may have a three-dimensional volumetric template comprising a sphere having a centroid and a radius. The three-dimensionally location of the centroid and radius are determined by analyzing multiple two-dimensional images taken at different angles.

Based upon a centroid and radius of the biological object or cell, controller 470 may model in three-dimensional space the size and internal depth/location of internal structures, such as the nucleus and organelles. For example, with respect to cells, controller 470 may utilize a predefined template of a cell to identify the cell wall and the nucleus. As indicated by block 518, using a predefined template, controller 470 additionally identifies regions or points of interest, such as organs or organelles of the cell. As indicated by block 524, controller 470 matches the centroid of the cell membrane, nucleus and organelles amongst or between the consecutive frames so as to estimate the relative movement (R, T) between the consecutive frames per block 528

As indicated by block 534, based upon the estimated relative movement between consecutive frames, controller 470 reconstructs the centroid coordinates in three-dimensional space. As indicated by block 538, the centroid three-dimensional coordinates reconstructed from every two frames are merged and aligned. A single copy of the same organelles is preserved. As indicated by block 542, controller 470 outputs a three-dimensional volumetric parametric model of object 52.

FIGS. 8-12 illustrate one example modeling process 600 that may be utilized by 3-D modeler 70 or controller 470 in the three-dimensional volumetric modeling of the biological object. FIG. 8-12 illustrate an example three-dimensional volumetric modeling of an individual cell. As should be appreciated, the modeling process depicted in FIGS. 8-12 may likewise be carried out with other biological objects.

As shown by FIG. 8, two-dimensional video/camera images or frames 604A, 604B and 604C (collectively referred to as frame 604) of the biological object 52 (schematically illustrated) are captured at different angles during rotation of object 52. In one implementation, the frame rate of the imager or camera is chosen such as the object is to rotate no more than 5° per frame by no less than 0.1°. In one implementation, a single camera captures each of the three frames during rotation of object 52 (schematically illustrated with three instances of the same camera at different angular positions about object 52) in other implementations, multiple cameras may be utilized.

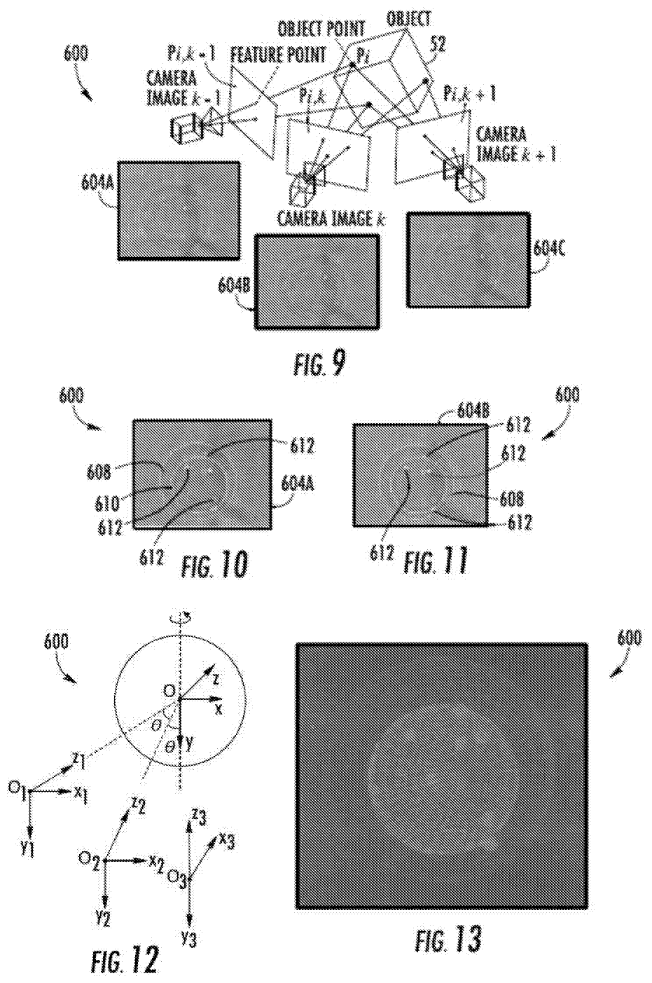

As shown by FIGS. 9 and 10, after image preprocessing set forth in block 508 in FIG. 5, edge detection, circle fitting another feature detection techniques are utilized to distinguish between distinct structures on the surface and within object 52, wherein the structures are further identified through the use of a predefined template for the object 52. For the example cell, controller 470 identifies wall 608, its nucleus 610 and internal points of interest, such as cell organs or organelles 612 in each of the frames (two of which are shown by FIGS. 10 and 11).

As shown by FIG. 12 and as described above with respect to blocks 524-538, controller 470 matches a centroid of a cell membrane, nucleus and organelles between consecutive frames, such as between frame 604A and 604B. Controller 470 further estimates a relative movement between the consecutive frames, reconstructs a centroid's coordinates in three-dimensional space and then utilizes the reconstructed centroid coordinates to merge and align the centroid coordinates from all of the frames. The relationship for the relative movement parameters R and T is derived, assuming that the rotation axis keeps still, and the speed is constant all the time. Then just the rotation speed is utilized to determine R and T (\vec{O₁O₂}), as shown in FIG. 10, where:

$$\vec{{O}_{1} {O}_{2}} = \vec{{OO}_{1}} \cdot {R}_{{θ}^{-}} \vec{{OO}_{1}} ;$$

$${R}_{θ} = {R}_{y} ( θ ) = [ \begin{matrix}cos θ & 0 & sin θ \\ 0 & 1 & 1 \\ - s in θ & 1 & cos θ\end{matrix} ]$$

based on the following assumptions:
θ is constant;
   |\vec{OO₁}|=|\vec{OO₂}|=|\vec{OO₃}|= . . . ;
rotation axis doesn't change (along y axis); and
\vec{OO₁} is known.
   As shown by FIG. 13, the above reconstruction by controller 470 results in the output of a parametric three-dimensional volumetric model of the object 52, shown as a cell.

Although the present disclosure has been described with reference to example implementations, workers skilled in the art will recognize that changes may be made in form and detail without departing from the spirit and scope of the claimed subject matter. For example, although different example implementations may have been described as including features providing one or more benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A cellular object growth and imaging system comprising:
a growth platform comprising:
a body having a cellular object fluid suspension region;
a media supply passage extending within the body to the cellular object fluid suspension region;
at least one fluid pump on the body to selectively deliver media to the cellular object fluid suspension region;
a waste discharge passage extending within the body from the cellular object fluid suspension region;
a cellular object rotator on the body adjacent the cellular object fluid suspension region to rotate a cellular object within the cellular object fluid suspension region, wherein the cellular object fluid suspension region permits optical imaging of the cellular object suspended in a fluid during rotation by the cellular object rotator.

2. The cellular object growth and imaging system of claim 1, wherein the waste discharge passage fluidly communicates with the cellular object fluid suspension region the at least one opening sized to inhibit entry of cellular objects within the cellular object fluid suspension region into the waste discharge passage.

3. The cellular object growth and imaging system of claim 1, wherein the waste discharge passage fluidly communicates with the cellular object fluid suspension region through at least one opening having at least one dimension less than or equal to 9 μm.

4. The cellular object growth and imaging system of claim 1, wherein the cellular object rotator comprises electrodes to form an electric field within the cellular object fluid suspension region.

5. The cellular object growth and imaging system of claim 1, wherein the cellular object rotator comprises a pair of spaced electrodes to form a nonuniform nonrotating electric field within the cellular object fluid suspension region to apply dielectrophoretic force to a cellular object within the cellular object fluid suspension region.

6. The cellular object growth and imaging system of claim 1, wherein the fluid pump comprises an inertial pump.

7. The cellular object growth and imaging system of claim 1, further comprising a second fluid pump on the body along the along the waste discharge passage to selectively deliver waste from the cellular object fluid suspension region.

8. The cellular object imaging growth and system of claim 1, wherein the media supply passage and the waste discharge passage are connected to an open space, the platform further comprising a controller to output control signals controlling supply of media to the cellular object fluid suspension region and the discharge of waste from the cellular object fluid suspension region so as to maintain a pendant drop of fluid suspended in the open space by surface tension, the pendant drop providing the cellular object fluid suspension region.

9. The cellular object growth and imaging system of claim 1, wherein cellular object fluid suspension region comprises a fluid chamber having a window through which imaging of a cellular object within the fluid chamber is provided.

10. The cellular object growth and imaging system of claim 1 further comprising a transparent pane across the window.

11. The cellular growth and object imaging system of claim 1 further comprising a gas supply to controllably supply a non-air gas to the cellular object fluid suspension region.

12. The cellular object growth and imaging system of claim 1, wherein the body has a second cellular object fluid suspension region, the platform further comprising:
a second media supply passage extending within the body to the second cellular object fluid suspension region;
a third fluid pump on the body along the second media supply passage to selectively deliver media to the second cellular object fluid suspension region;
a second waste discharge passage extending within the body from the second cellular object fluid suspension region;
a second fluid pump on the body along the along the waste discharge passage to selectively deliver waste from the second cellular object fluid suspension region; and
a second cellular object rotator on the body adjacent the second cellular object fluid suspension region to rotate a second cellular object within the second cellular object fluid suspension region, wherein the second cellular object fluid suspension region permits optical imaging of the second cellular objects suspended in a fluid during rotation by the second cellular object rotator.

13. A cellular object growth and imaging method comprising:
providing a cellular object fluid suspension region of a growth platform with a cellular object;
controllably pumping media to the cellular object fluid suspension region with at least one pump on the growth platform;
controllably pumping waste from the cellular object fluid suspension region with the at least one pump on the growth platform;
rotating the cellular object within the cellular object fluid suspension region; and
imaging the cellular object during rotation within the cellular object fluid suspension region.

14. The cellular object growth and imaging method of claim 13 further comprising forming a pendant drop suspended from the body by surface tension, wherein the pendant drop forms the cellular object fluid suspension region and wherein providing the cellular object comprises injecting the cellular object into the pendant drop.

15. A non-transitory computer-readable medium containing instructions to direct a processing unit to:
output control signals to a pump on a growth platform to supply fluid media to a cellular object fluid suspension region of the growth platform;
output control signals causing waste to be discharged from the cellular object fluid suspension region; and
output control signals to electrodes on the growth platform to form an electric field within the cellular object fluid suspension region to rotate the cellular object for imaging.
